(19) United States
(12) Patent Application Publication
(10) Pub. No.: US 2002/0157141 A1
(43) Pub. Date: Oct. 24, 2002

(54) Growth medium and process for making wine

(75) Inventor: Johann-Joachim Pangratz, Dorsten (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: DEGUSSA AG, Duesseldorf (DE)

(21) Appl. No.: 10/112,867

(22) Filed: Apr. 2, 2002

(30) Foreign Application Priority Data
Apr. 2, 2001 (DE) 101 16 442.4

(51) Int. Cl. A01H 5/00; C12G 1/00

(52) U.S. Cl. 800/298; 426/15

(57) ABSTRACT

A growth medium for organism such as plants including fruits, vegetables, etc. and a process for the efficient production of plants such a grapes by providing a supply of water to both newly planted and previously productive plants, thereby improving the efficiency of growth, fruit and plant production, and wine production.

### BACKGROUND OF INVENTION

1. Field of the Invention

The present invention relates to a growth medium for plants such as fruits, vegetables, etc. and to a process for the efficient production of plants such as grapes by providing a supply of water to both newly planted and previously productive plants, thereby improving the efficiency of growth, fruit and plant production, and wine production.

2. Discussion of the Background

A process for making wine from cultivation to decantation of the wine into containers is previously known (see B. Götz, Weinbau, Stuttgart 1987). The stock and row distances during planting of the vineyard depend on the terrain of the land. Therefore, the ground may be prepared by leveling off, deep-plowing (trenching), plowing and marking out with stakes. Planting holes are then established in the ground. The graft vines are set into planting holes either by hand or automatically. Automatic planting of vines can be achieved by using a planting machine to bore holes in the ground. To guarantee good growth and development of a productive root ball of the vines, they must be watered during or immediately after being set into the ground. Watering must be repeated over an extended period of time if required so that they do not dry out.

After two years from the time the vines have been set into the ground, the first harvest of grapes is anticipated. In the spring, before the buds burst, the grapes are cut appropriately. After budding, the grapes are tied to wire support frames. Then, the ripe grapes are harvested or picked according to each type of grape. Depending on the grape, the harvesting occurs sometime between August and October. After harvest, the picked grapes are crushed, pressed, and squeezed into a must, or juice. The pressed must, or juice, is then fermented. Culture yeast may be added to the fermenting must after pressing. In cases of red wine production, fermentation of the grape must occur after crushing. Pressing then takes after fermentation. The yeast is then racked off, or separated. The wine matures during fermentation and racking off the yeast. After maturing the must into wine, unfermented reserves of grape must may be added if necessary. Unfermented reserves of grape must are added to adjust the residual sugar content of the resulting wine, the wine is decanted into containers to be sold. Examples of containers are bottles and barrels.

A vital step of the winemaking process is the irrigation of the vineyards. Newly planted vineyards require adequate water supply in the early months and years of development to ensure an adequate supply of nutrients so that their root systems may develop. Further, previously-productive vineyards require adequate water supply. Normally, the rainfall necessary to adequately supply both newly planted and previously-productive vineyards is more than 400 mm of annual precipitation. If the annual precipitation is less than 400 mm, irrigation must be performed in areas having sufficient surface water sources nearby. Examples of such sources of water include rivers and lakes. Irrigation, however, is very costly.

Sprinkler units may be used for irrigating newly planted and previously-productive vineyards. Sprinkler units may include pipe systems, which provide water from overhead. Also, dribble irrigation may be used for irrigating newly planted and previously-productive vineyards. Dribble irrigation may include hose systems. Both pipe and hose systems, however, rely on adequate sufficient surface water sources be nearby. Therefore, sufficient surface water sources must be near the vineyard. If none is nearby, then the vineyards must be stock watered, where water from transport containers is carried by hand and distributed throughout the vineyard.

During the course of time, the yields from harvesting productive vineyards declines. This is attributable to a shortage in water supply, as well as a shortage of nutrients. Normally, the shortage of water and the shortage of nutrients are directly related and co-dependant. It is normally more costly to irrigate a productive vineyard that has declining yields from harvest than it is to clear out and replace it. However, clearing out and replacing productive vineyards requires high investment costs. Further, the artificial irrigation of newly planted vineyards is very expensive.

### SUMMARY OF THE INVENTION

One object of the present invention is a growth medium comprising soil and a superabsorbent polymer.

Another object of the present invention is a process of growing organisms such as plants in a growth medium comprising soil and a superabsorbent polymer.

Another object of the present invention is a process of growing plants in a growth medium comprising soil and a superabsorbent polymer.

Another object of the present invention is a process of growing fruits and vegetables in a growth medium comprising soil and a superabsorbent polymer.

Another object of the present invention is a method for making wine, in which the ground is prepared for planting with a superabsorbent polymer, the vines are planted and watered, the grapes are picked and pressed, the resulting must is fermented, converted into wine, and the wine is decanted into containers.

Another object of the present invention is a process for producing grapes and currants by means of viniculture, where the ground is prepared for planting with a superabsorbent polymer, the vines are planted and watered and the grapes are picked.

Another object of the present invention is a process for supplying water to vines during cultivation of the vines.

Another object of the present invention is a process for making grapes by viniculture, in which the ground is prepared for planting, the vines are planted and watered, and the grapes are picked, wherein a superabsorbent polymer is introduced into the ground.

Another object of the present invention is a process for supplying water to vines during viniculture, which is characterized in that a superabsorbent polymer is introduced into the ground.

Another object of the present invention is a method for producing raisins.

Another object of the present invention is a method for producing currants.

### DETAILED DESCRIPTION OF THE PRESENT INVENTION

Unless specifically defined, all technical and scientific terms used herein have the same meaning as commonly understood by a skilled artisan in biochemistry, chemistry, and materials science.

Methods and materials similar or equivalent to those described herein can be used in the practice or testing of the present invention, and suitable methods and materials are described herein. All publications, patent applications, patents, and other references mentioned herein are incorporated by reference in their entirety. In case of conflict, the present specification, including definitions, will control. Further, the materials, methods, and examples are illustrative only and are not intended to be limiting.

The present invention relates to a growth medium comprising soil and a superabsorbent polymer. This growth medium may be used to grow organisms because it provides water that is readily available for a sustained period of time during organism growth. Examples of organisms that may be grown in or on the growth medium include plants. An example of a plant that may be grown on or in the growth medium include grapes and currants.

The present invention relates to a process for making wine, in which newly planted and previously-productive vineyards are protected from damage caused by insufficient water supply. One cause of the insufficient water supply may be minimal precipitation, or dryness, in cultivated areas. Therefore, newly planted and previously-productive vineyards are provided with longer, more useful lives during times of dryness in the advanced stage of the vineyard's growth. The present invention discloses a process by which the above-mentioned negative effects of dryness are reduced.

Introduction of superabsorbent polymers into the ground in the vicinity of newly planted vineyards reduces the danger of dryness to such vineyards. This is due to an improved water and nutrient supply. Therefore, the life of a vineyard may be substantially prolonged by such introduction of superabsorbent polymers into the ground, and the cost of replacing the vineyard is prolonged.

Superabsorbent polymers are chemical substances that absorb large quantities of water. Further, superabsorbent polymers can slowly release the absorbed water over a delayed period of time. An example of a superabsorbent that can be used according to the present invention are copolymers of acrylic acid derivatives or the polymer is preferably of a synthetic type and water soluble.

Examples of applicable polymers are water soluble carboxylic polymers such as polyacrylic acid and its salts, polymethacrylic acid and its salts, copolymers of acrylic acids or methacrylic acids with co-monomers such as alkyl acrylates, alkyl methacrylates, and polymaleic acids and their salts. The preferred salts are alkali metal salts such as for example sodium. Further, particularly preferable salts include potassium or ammonium salt thereof. These superabsorbent polymers may have a weight average molecular weight of from about 60,000 to about 2,000,000 or higher with a molecular weight of from 60,000 to 500,000 preferred, and 100,000 to 300,000 most preferred. The polymers may be used in the acid or the neutralized form. Preferably, the polymer should also be anionic. The acrylic acid can be a potassium or ammonium salt thereof.

Preferably, the crosslinked and non-crosslinked acrylic acid copolymers are very good superabsorbent polymers. One example of an acrylic acid polymer that may be used in the present invention has the following formula:

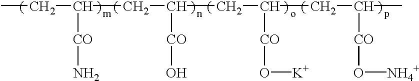

Further examples of the superabsorbent polymer include partially neutralized, slightly crosslinked polymers and copolymers of acrylic acid and acrylamide, graft polymerizates of starch, as well as crosslinked starches and cellulose derivatives. The grain size distribution of the powdered superabsorbent polymer employed may vary within wide limits. The grain size distribution may range from 0.1 μm to up to 20,000 μm are possible. Preferred grain fractions are in the range of from 1 μm to up to 5,000 μm. Particularly preferred are grain fractions ranging from 20 to 1,000 μm.

One example of a commercially available superabsorbent polymer is STOCKOSORB® AGRO by Stockhausen, Krefeld. Other examples include HYSORB® products by BASF, Ludwigshafen.

The superabsorbent polymers according to the present invention may be in the form of a dry polymerizate, or in the form of a gel when in contact with water. The dry polymerizate is a three-dimensional network of the superabsorbent polymer.

Suitable quantities of superabsorbent polymers can be introduced on the surface of the ground in granulate form during the preparation of the ground prior to planting the vineyard. Further, suitable quantities of superabsorbent polymers can be introduced into the surface of the ground. A method to introduce a superabsorbent polymer into the ground includes the injection of an aqueous superabsorbent polymer suspension. The injection method utilizes a lance system of injecting the superabsorbent polymer into the ground in the vicinity of the roots of vines. This method substantially reduces the risk of vineyards drying out.

The superabsorbent polymer can be introduced into the ground for agriculture, including viniculture, in the vicinity of the vine roots of newly planted and previously productive vineyards. The superabsorbent polymer is introduced into the ground preferable in quantities of from 1 kg to 10 kg per m³ of soil. The ranges for the quantities of superabsorbent polymer include all specific values and subranges therebetween, such as 1.5, 2, 2.5, 3, 3.5, 4, 4.5, 5, 5.5, 6, 6.5, 7, 7.5, 8, 8.5, 9, and 9.5 kg per m³ of soil.

Superabsorbent polymers may decompose after 4 or 5 years of being in the ground. By this time, the young plants of newly planted vineyards have already developed substantial sturdy root systems that penetrated the ground. Such root systems are sufficient enough to survive dryness by adequately ensuring a supply of water, even in the absence of superabsorbent polymer after it decomposes. However, at any time the superabsorbent polymer or more superabsorbent polymer can be added to the soil around the root systems.

The soil of the ground may be mixed with the superabsorbent polymer directly. Further, the soil of the ground may be blended with superabsorbent polymer granulate. After digging a hole in the ground, the soil and superabsorbent polymer granulate mixture may then be placed into the hole. The superabsorbent polymer granulate may also be plowed into the ground.

The superabsorbent polymer can be introduced at an appropriate depth into the ground as an aqueous suspension. The aqueous suspension is preferably introduced into the ground using a lance system. This method is particularly preferred when introducing the superabsorbent polymer to previously productive vineyards. Tractor drawn sprinkler systems intended for spreading aqueous systems of chemicals intended to protect plants may also be used as lance systems. These sprinkler systems have a pump that is capable of generating a pressure of up to 40 bar or greater. A pipe can be connected to the pump. The pipe may be approximately 1.5 meters in length, and may have an end nozzle and a hand lever valve. The hand lever valve is used to release the water flow. The pipe may be attached to the pump on the side with pressure. Similarly, a hose may be connected to the pump.

The present invention relates to a method of producing raisins. When grapes reach maturity after growing in a growth medium of soil and a superabsorbent polymer, they may be harvested and placed in the sun to dry to produce raisins. Once they dehydrate, the grapes may be collected and stored until their final production and packing process as raisins.

The present invention further relates to a method of producing currants. Examples of currants include those from the genus Ribes. Specific species from the genus Ribes may include Ribes rubrum, Ribes petraeum, Ribes sativum, Ribes nigrum, and Ribes ussuriense, for example. With their fibrous, shallow roots, the growth medium of the present invention may readily be provided by planting them in the growth medium. Further, the above-described dribble irrigation may be used to apply superabsorbent polymer to soil in the vicinity of the roots of currants, thereby providing the growth medium and an adequate supply of water until the fruit is harvested.

The present invention is explained in more detail with the aid of the following embodiment examples. As can be seen from the following examples, the process according to the present invention can significantly prolong the life of newly planted and previously productive vineyards.

### EXAMPLES

Numerous modifications and variations on the present invention are possible in light of the above teachings. The following embodiment examples are in no way intended to narrow the scope of the teachings described above. Alternatively, the following examples demonstrate that the present invention can significantly prolong the life of newly planted and previously productive vineyards.

### Example 1

According to the present invention, the superabsorbent polymer STOCKOSORB® AGRO by Stockhausen, Krefeld is plowed into the ground in a quantity of 3 kg per m³ of soil during ground preparation of a newly planted vineyard. Water is supplied to the vineyard through means of natural precipitation such as rain. Alternatively, the required water may be artificially supplied by sprinkling or any other form of introducing water into the root region of the vines. The water is stored on the superabsorbent polymer. Therefore, the water no longer runs off into the ground water because of deficient storage potential in the ground itself. Instead, the water stored on the superabsorbent polymer is now available to the vines. A comparison with a neighboring parcel of land of similar size, in which no superabsorbent polymer has been introduced into the ground, shows that when the process according to the present invention is applied, approximately 30% fewer vines dry out. Therefore, additional irrigation is required to prevent the vines from drying out in the neighboring parcel without superabsorbent polymer in the ground.

### Example 2

A productive vineyard must be cleared out after approximately 20 to 25 years and be replaced by a newly planted vineyard because the yield of grapes per hectare has fallen below the economical threshold of efficiency. Planting a new vineyard is a costly investment, which can be postponed by 4 to 5 years if the superabsorbent polymer STOCKOSORB® AGRO by Stockhausen, Krefeld is introduced as an aqueous suspension in a quantity of 3 kg per m³ soil by means of a lance system into the root area of the productive vines.

The present application claims priority to German Application No. DE 101 16 442.4, filed on Apr. 2, 2001, which is hereby incorporated by reference in its entirety.

Numerous modifications and variations on the present invention are possible in light of the above teachings. It is, therefore, to be understood that within the scope of the accompanying claims, the invention may be practiced otherwise than as specifically described herein.

What is claimed is:

1. A grapevine planted in a soil, wherein the soil comprises a superabsorbent polymer.

2. The grapevine according to claim 1, wherein the soil and the superabsorbent polymer are present at a ratio of from 1 to 10 kg of superabsorbent polymer for every 1 m³ of soil.

3. The grapevine according to claim 1, wherein the superabsorbent polymer comprises a copolymer of an acrylic acid derivative.

4. The grapevine according to claim 3, wherein the acrylic acid derivative is a potassium or ammonium salt thereof.

5. The grapevine according to claim 1, wherein the superabsorbent polymer is in the form of a granulate.

6. The grapevine according to claim 1, wherein the superabsorbent polymer is provided as an aqueous suspension.

7. A process of growing a grapevine, comprising
planting the grapevine in a soil comprising a superabsorbent polymer.

8. The process according to claim 7, wherein the superabsorbent polymer is provided as an aqueous suspension.

9. A method of producing raisins, comprising
growing a grapevine according to the process according to claim 7.

10. A process of making a growth medium for a grapevine, comprising mixing soil and a superabsorbent polymer.

11. The process according to claim 10, further comprising mixing the soil and the superabsorbent polymer in a ratio of from 1 to 10 kg of superabsorbent polymer for every 1 m³ of soil.

12. The process according to claim 10, wherein the superabsorbent polymer comprises a copolymer of an acrylic acid derivative.

13. The process according to claim 12, wherein the acrylic acid derivative is a potassium or ammonium salt thereof.

14. The process according to claim 10, wherein the superabsorbent polymer is in the form of a granulate.

15. A process of growing a grapevine, comprising
plowing a superabsorbent polymer into the ground;
planting a grapevine in the ground; and
watering the grapevine.

16. The process according to claim 15, wherein the superabsorbent polymer comprises a copolymer of an acrylic acid derivative.

17. The process according to claim 16, wherein the acrylic acid derivative is a potassium or ammonium salt thereof.

18. The process according to claim 15, wherein the superabsorbent polymer is in the form of a granulate.

19. The process according to claim 15, wherein the superabsorbent polymer is provided as an aqueous suspension.

20. A process of growing a grapevine, comprising
planting a grapevine in a soil;
watering the plant; and
introducing a superabsorbent polymer into the soil subsequently or simultaneously to the planting or watering.

21. The process according to claim 20, comprising
digging a planting hole;
placing the superabsorbent polymer in the planting hole; and
subsequently or simultaneously placing soil in the planting hole, prior to planting the grapevine.

22. The process according to claim 20, further comprising introducing the superabsorbent polymer at a ratio of from 1 to 10 kg of the superabsorbent polymer for every 1 m³ of soil.

23. The process according to claim 20, wherein the superabsorbent polymer comprises a copolymer of an acrylic acid derivative.

24. The process according to claim 23, wherein the acrylic acid derivative is a potassium or ammonium salt thereof.

25. The process according to claim 20, wherein the superabsorbent polymer is in the form of a granulate.

26. The process according to claim 20, wherein the superabsorbent polymer is provided as an aqueous suspension.

27. The process according to claim 20, further comprising introducing the superabsorbent polymer into the soil by plowing.

28. The process according to claim 20, comprising introducing an aqueous superabsorbent polymer suspension into the soil by means of a lance system.

29. The process according to claim 20, wherein the grapevine is in a vineyard.

30. The process according to claim 29, wherein the vineyard is a newly planted vineyard.

31. The process according to claim 29, wherein the vineyard is a productive vineyard.

32. A method of producing wine, comprising growing a grapevine according to the process of claim 20.

33. The method according to claim 32, further comprising
picking a grape from the grapevine;
pressing the grape into a must;
fermenting the resulting must;
maturing the must into the wine; and
decanting the wine into containers.

34. A method of producing a currant, comprising
planting a plant capable of producing a currant in a soil comprising a superabsorbent polymer.
